(19) United States
(12) Patent Application Publication
(10) Pub. No.: US 2004/0172036 A1
(43) Pub. Date: Sep. 2, 2004

(54) Angled acetabular reamer and method of use

(76) Inventor: Donald Dye, Pflugerville, TX (US)

Correspondence Address: ZIMMER TECHNOLOGY, INC., 150 N. WACKER DRIVE, SUITE 1200, CHICAGO, IL, 60606, US

(21) Appl. No.: 10/375,324

(22) Filed: Feb. 27, 2003

(51) Int. Cl. A61B 17/16

(52) U.S. Cl. 606/81

(57) ABSTRACT

A method and apparatus for performing minimally invasive hip surgery to implant an acetabular shell into the acetabulum. An angled acetabular reamer includes a handle section and a reaming section that is disposed at an angle with respect to the handle section. The reaming section is positioned through the minimally invasive incision to ream the acetabulum.

### FIELD OF THE INVENTION

The disclosure herein generally relates to a method and apparatus for performing minimally invasive hip replacement surgery for the acetabulum using an angled acetabular reamer.

### BACKGROUND OF THE INVENTION

Traditional hip replacement surgery has been used in the United States since as early as the 1960's. The surgical technique to implant a hip has not drastically changed over the years, and today, this technique is quite successful. In fact, the surgical technique is prolifically used throughout the world and has a known success rate of over 90%. Certainly, the traditional surgical technique is fundamentally sound and predictable.

Unfortunately, traditional techniques to implant a hip have well recognized shortcomings. Most importantly, a rather large incision is made on the side of the hip. The incision can extend from 6 to 12 inches; the actual length of the incision depends on the size of the patient and type of surgery (revision versus total hip arthroplasty, for example). A long, deep incision can divide a number of important stabilizing muscles and tendons and further damage the hip joint and surrounding soft tissue. Inevitably, long incisions lead to larger blood losses, longer rehabilitation times for patients, and unsightly scar lines. A patient can easily spend four or five days in the hospital after a total hip arthroplasty, for example.

Recently, surgeons have been developing new, less invasive surgical techniques to perform total hip arthroplasty and revision hip surgery. Minimally invasive surgery, or MIS, is one such technique with great promise to become a popular and accepted technique for implanting a hip.

MIS has significant advantages over traditional hip replacement surgery. Most importantly, a rather small incision is made on the side on the hip. This incision is approximately 3 to 5 inches long, and the benefits of a shorter incision are enormous.

First and foremost, the patient can recover in a much shorter period of time after a MIS. The recuperation time in the hospital can be a few days and significantly reduce the cost to both the patient and hospital. In fact, some patients are leaving the hospital within 24 to 48 hours after the surgery. Obviously, this shortened time period is extremely important to the patient.

As another advantage, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Presently, instruments to perform MIS are being developed and refined. These instruments have a vital role in the ability to perform a successful minimally invasive surgery. These instruments, for example, must enable the surgeon to place the hip implant in a very precise location. If the implant is not accurately placed, then complications, such as dislocation or subluxation, can occur. Further and most importantly, the instruments must consistently and reliably perform through a small three inch opening in the patient.

A successful design of instruments for MIS has other challenges as well. Specifically, the instrument must be easy to use and facilitate the implantation procedure. If the MIS instrumentation is too cumbersome or not easy to manipulate, then the surgeon will be less likely to use minimally invasive surgery. The patient, then, will not reap the benefits MIS has to offer.

As yet another consideration, MIS instrumentation must appeal to a wide range of orthopedic surgeons with various skills and experience. If, for example, the instruments are too complex and complicated, then they will not be appealing and accepted in the orthopedic surgical community. Further yet, the training and skill level required to use the instruments and become proficient with them, cannot be overly taxing on the orthopedic surgeons.

In short, instruments, and in particular acetabular reamers, play a vital role in MIS surgery for hip implantation. It therefore would be advantageous to provide a new method and accompanying instruments for performing a minimally invasive surgery to implant a prosthetic hip.

### SUMMARY OF THE INVENTION

The present invention is directed to a method and apparatus for performing minimally invasive hip surgery and, more particularly, to an improved method and apparatus for performing minimally invasive hip replacement surgery for the acetabulum using an angled acetabular reamer.

The method of the present invention generally comprises the steps of templating the acetabulum to estimate the size of reamer and acetabular components; incising the surgical site with a single incision approximately three inches in length; exposing the acetabular joint and dislocating the hip from the acetabulum; providing an angled acetabular reamer; reaming the acetabulum with the angled reamer; providing an acetabular shell impaction instrument; inserting and aligning a trial shell into the reamed acetabulum; inserting a trial insert to the trial shell; removing the trial insert and shell; inserting and aligning an implant shell into the reamed acetabulum; impacting the implant shell with the acetabular shell impaction instrument; inserting and impacting an implant insert into the implant shell; and closing the surgical site.

The angled acetabulum reamer of the present invention generally comprises a body having two sections, a handle section and a reaming section. The handle section is adapted to be gripped with a hand and includes a proximal end that connects with an automated driving instrument. The reaming section has distal reaming end adapted to engage and connect with an acetabular reamer. Most importantly, the reaming section is formed at an angle with respect to the handle section. This angle provides numerous advantages to the angled acetabular reamer and makes it ideal for use in minimally invasive orthopedic hip surgery. An elbow or sleeve connects the handle and reaming sections. This elbow houses a coupling mechanism having a “dogbone” shape. The coupling mechanism transfers torque from a driving shaft (housed in the handle section) to a driven shaft (housed in the reaming section). Preferably, the reaming section is at about a 45° angle with respect to the handle section.

One important advantage of the present invention is that the method and acetabular reamer are used in a minimally invasive orthopedic hip surgery. A single, small three inch incision is made at the surgical site on the side on the hip. The method of the present invention, thus, enjoys the benefits of a shorter incision compared to traditional hip surgery that uses a much longer incision. As one benefit, the patient can recover in a much shorter period of time after a MIS. The recuperation time in the hospital can be a few days and significantly reduce the cost to both the patient and hospital. This shortened time period is extremely important to the patient. Further, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Another important advantage of the present invention is that an angled acetabular reamer is used. This reamer is specifically designed and adapted to be used in minimally invasive surgical techniques for reaming the natural acetabulum of a patient. The angle between reaming section and handle section provides the correct angular orientation to ream the acetabulum. As such, the correct angulation and anteversion can be reamed. In use, the reaming section of the angled reamer is positioned through the MIS incision and into the surgical site so the reamer or cutter can engage the natural acetabulum. Simultaneously, the handle section protrudes out from the incision and away from the edges of the wound.

Another important advantage of the present invention is that the angulation of the reaming section keeps the handle away from the entrance to the surgical site. In MIS, it is particularly important to maintain a clear and unobstructed access to the surgical site since it is so small, measuring approximately 3-5 inches in length. Further, it is important not to disrupt or aggravate the sides of the wound channel during the surgical procedure. In the present invention, the handle extends outwardly and away from the sides of the surgical site. The instrument, thus, can be used without unnecessarily disrupting or aggravating the surgical site.

As another advantage, the coupling mechanism that couples the drive shafts smoothly and consistently transfers torque at a constant velocity down the drive shaft to the reamer. Further, the coupling mechanism provides a short reaming section. This shorter section makes the reamer easier to manipulate in the small surgical incision and easier to control during reaming.

The reamer of the present invention enables the surgeon to ream precisely the natural acetabulum at the correct angular orientation. As such, the shell can be accurately placed. The likelihood of a complication associated with an incorrectly placed acetabular implant is reduced.

As another advantage, the acetabular reamer can consistently and reliably perform through a small three inch opening in the patient. The coupling mechanism employs a limited number of components and moving parts.

Further, a driving guide may be attached to the handle section of the angled reamer. This driving guide assists the surgeon in guiding the reamer at the proper angulation into the acetabulum.

Further yet, the instrument is easy to use and facilitates the implantation procedure. As such, the acetabular reamer can appeal to a wide range of orthopedic surgeons with various skills and experience. Further yet, the training and skill level required to use the instrument and become proficient with it is not overly taxing on the orthopedic surgeon.

### BRIEF DESCRIPTION OF THE DRAWINGS

FIG. 1 is a view of a patient showing a femur and femoral head positioned in the acetabulum with an MIS incision marked along the hip.

FIG. 2 is a view of an angled acetabular reamer of the present invention reaming an acetabulum with a reamer through a MIS surgical site.

FIG. 3 is a view of an angled instrument engaged with a screw-hole plug connectable to the shell embedded in the acetabulum.

FIG. 4 is a view of a flexible drill drilling a hole in the acetabulum to receive a bone screw.

FIG. 5 is a view of a shell embedded in the acetabulum with a bone screw and dome plug insert into the shell.

FIG. 6 is a view of an insert being inserted into an acetabular shell embedded in the acetabulum.

FIG. 7 is a perspective view of the angled acetabular reamer of the present invention.

FIG. 8 is a top view of the reamer of FIG. 7.

FIG. 9 is an end view of the reamer of FIG. 7.

FIG. 10 is a view showing the coupling mechanism between the driving shaft and driven shaft in the elbow of the angled acetabular reamer.

FIG. 11 is a top view of the dogbone coupling mechanism.

FIG. 12 is a side view of the dogbone coupling mechanism.

### DETAILED DESCRIPTION

The instruments, method, and steps of the present invention are now described in more detail. The method describes the steps to perform a minimally invasive surgery to implant a prosthetic acetabular component into the natural acetabulum of a patient. Some of these steps described in the method are known to those skilled in the art and will not be discussed in great detail. Further, one skilled in the art will appreciate that certain steps may be altered or omitted while other steps may be added without departing from the scope of the invention. The novel steps of the present invention, for example, can be applied to total hip arthroplasty, to revision surgeries for total and partial hip replacement, and to other orthopedic surgeries using minimally invasive surgical techniques.

To facilitate a discussion of the present invention, the method of implanting a prosthetic acetabular component is divided into a plurality of steps or sections. Each of these sections is discussed seriatim.

More specifically, the method of the present invention teaches how to implant a prosthetic acetabular shell and insert into the natural acetabulum using an angled acetabular reamer to ream the acetabulum through a small MIS incision. For illustrative purposes, the discussion focuses on implanting a Converge™ Acetabular System of Centerpulse Orthopedics Inc. of Austin, Tex. This system illustrates one possible acetabular system that can be used. One skilled in the art will appreciate that other, different acetabular systems can also be used with the method and apparatus of the present invention without departing from the scope of the invention.

### Templating the Acetabulum

Typically, the side of the acetabulum to be reconstructed is templated. Use of a template enables the surgeon to make an estimation of the size of reamers to be used and the size of acetabular component to be inserted. The acetabulum is templated on the both the anterior-posterior (A/P) and lateral radiographs. The hemisphere of the acetabular component is aligned with the mouth of the bony, natural acetabulum while simultaneously avoiding any osteophytes. On the A/P radiograph, the acetabular component should rest on the floor of the cotyloid notch and may touch the illoischial line. Further, the component should have a maximum lateral opening of about 40°. On the groin lateral radiograph, the cup size selected should contact the anterior and posterior rim of the bony, natural acetabulum and the medial subchondral bone. A correct position of the acetabular component will anatomically reproduce the center of rotation of the femoral head. If a bony defect is identified, use the correctly placed template to measure for proper size of the acetabular component and determine any need for bone graft.

### Incising the Surgical Site (See FIG. 1)

A relatively small, single minimally invasive incision is made at the surgical site. A minimally invasive incision for this procedure has a length from about 2½ inches to about 4 or 5 inches. The incision is slightly curved or straight, commences near the vastus tubercle, and continues toward the greater trochanter and posterior inferior spine. The incision should be carried down through subcutaneous tissue and fascia lata. Any muscle tissue should be gently split in line with its fibers. Retractors can now be used, as preferred, to retract portions of the site. At this time, a leg length measurement can be taken using techniques known in the art.

### Providing Retractors

The retractors have an elongated, flat, thin body with two primary sections, a handle section and a retracting section. The handle section is elongated and adapted to be gripped with a hand. A smooth curved section transitions the handle section to the retracting section. The retracting section has a paddle with a prong that curves outwardly and away from the paddle and handle section.

### Exposing the Acetabular Joint & Dislocating the Hip from the Acetabulum

Next, the knee is flexed, and the leg is internally rotated. Using a hot knife, the piriformis, short external rotators, quadratus femoris, and some posterior capsule are incised off the posterior trochanter to expose the lesser trochanter. Dislocation of the hip can now occur. A bone hook or skid may be used to avoid excess torsion on the femoral shaft.

At this time, retractors may be placed, for example under the femoral head or lesser trochanter, in order to achieve visualization for proper transection of the femoral neck if this procedure is desired at this time. If such transection occurs, the femoral neck should be transected at the templated level. Then retract the femur in an anterior direction to expose the acetabulum. Care should be taken to protect the sciatic nerve.

A retractor can be placed on the pelvis to hold the femur in an anterior position to the acetabulum. The capsule can be retracted in the posterior using retractors or pins. After the labrum and osteophytes are removed, at least a partial view of the acetabulum should be available.

### Providing an Acetabular Reamer (See FIGS. 7-12)

An acetabular reamer is provided to ream the natural acetabulum. The reamer is designed and adapted to be used with minimally invasive surgical techniques of the acetabulum. Specifically, the reaming section is shaped to fit through the small incision at the surgical site. Further, the reamer is angled so the distal end properly engages the natural acetabulum with the correct angular orientation and without disrupting the incision and surrounding soft tissue. The angled acetabular reamer of the present invention is more fully described with reference to FIGS. 7-12.

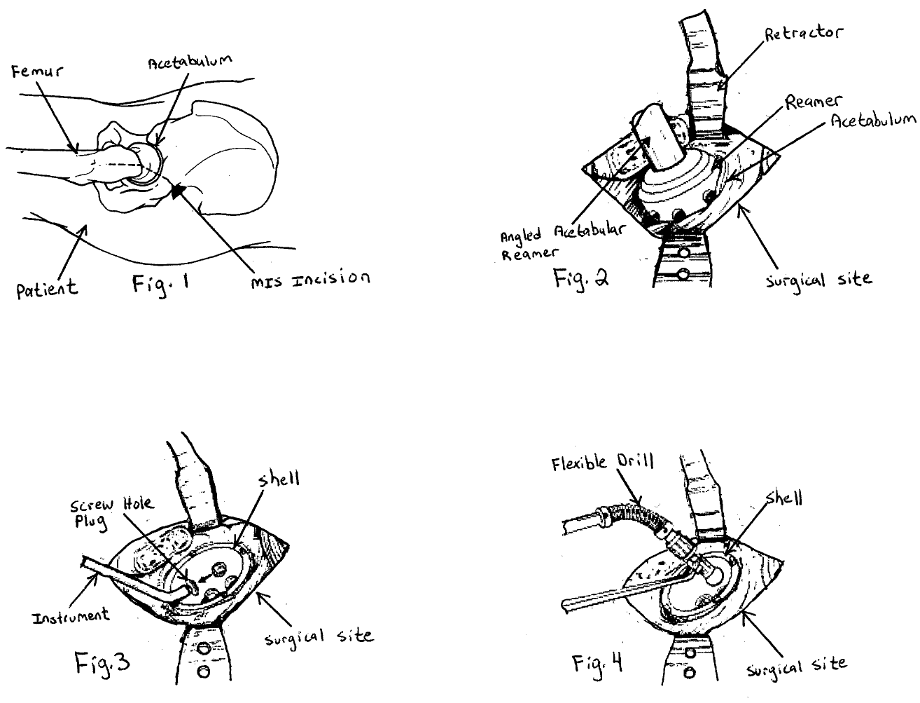

### Reaming the Acetabulum (See FIG. 2)

Reaming of the acetabulum should begin with a reamer that is two sizes smaller than the preoperatively selected acetabular component size. A smaller reamer ensures that the fit does not exceed the anterior-posterior diameter. Of course, the reamer should not be so small that excessive anterior or posterior reaming occurs.

After an appropriately sized reamer is connected to the acetabular reamer, reaming should begin transversely toward the cotyloid notch. The ridges of the horseshoe (or medial osteophytes) should be removed. Reaming then continues in the position of desired anteversion while simultaneously creating a hemisphere. Larger reamers are used until the anterior and posterior rim of the acetabulum is contacted. The reamer should not be sunk below the superior rim of the bony acetabulum or reamed through the cortical bone of the cotyloid notch. Cancellous bone will be evident where the horseshoe ridges have been removed. The proper size trial shell should be selected according to the size of the reamer.

### Providing an Acetabular Shell Impaction Instrument

An acetabular shell impaction instrument is provided to align and then impact the acetabular shell into the natural acetabulum. The instrument is designed and adapted to be used with minimally invasive surgical techniques of the acetabulum. Specifically, the instrument has a curved shape to fit through the small incision at the surgical site and precisely impact the implanted shell at the correct angular orientation. Further, this curvature enables the instrument to engage the shell in the acetabulum without disrupting the incision and surrounding soft tissue. Further yet, the instrument is adapted to move and align the acetabular shell while it is positioned in the acetabulum. It is important to position properly the shell before it is impacted and permanently seated in the acetabulum.

### Inserting a Trial Shell into the Acetabulum

The acetabular shell impaction instrument keys off the dome of the trial shell and is threaded or engaged in place. The instrument may offer anteversion and abduction references and rotational control. Preferably, the distal end of the instrument is adapted to mate with both the trial shell and implant shell in one single orientation. To connect the components, the distal end of the instrument is keyed and threadably attached to the trial shell. One skilled in the art will appreciate that the instrument, inserts, and shells can connect in various ways.

After the trial shell is inserted into the acetabulum, its position is verified through a trial window. The edge of the trial shell should be level with the anterior-inferior margins of the acetabulum and should completely fill the anterior-posterior bony acetabulum. The instrument can be used to move and align shell while it is positioned in the acetabulum. At this time, the trial shell can be manually tested to ensure that it is stable. If the trial is loose, then use the next larger size. If the trial is too tight, then ream the rim of the acetabulum. Importantly, the trial shell should be stable before selecting a similarly sized acetabular implant shell.

### Inserting a Trial Insert into the Trial Shell

Now, the trial insert is ready to be placed in the trial shell. An instrument is engaged in the rim of the trial insert and it is positioned inside the cavity of the trial shell. The trial insert contains a captured screw at the apex and can be threaded into the dome of the trial shell with a screwdriver or other tool. The trial components should be checked for proper fit and size.

At this point, the trials are removed from the surgical site. One skilled in the art, though, will appreciate that the trials could be temporarily left inserted to the natural acetabulum to articulate with a trial femoral prosthesis in a total hip replacement surgery.

### Inserting an Implant Shell into the Acetabulum

Some implant shells may be provided with flared rims and outer bone engaging spikes. In order to insert such a shell, cancellous bone slurry may be added within the acetabulum to fill existing bone cysts and provide an interface layer. Addition of this slurry typically occurs in total hip arthroplasty situations.

The acetabular implant shell is positioned into the acetabulum using the same acetabular shell impaction instrument used with the trial shell. Specifically, the distal connection end of the instrument is engaged and connected to the shell. The shell is partially inserted into the acetabulum until the rim begins to engage bone. The implant is then positioned with the instrument to the desired angular orientation, such as abduction and anteversion. Preferably, the shell is positioned with 20° to 25° of anteversion and with an abduction angle of about 35° to 45°. The anteversion can be verified using techniques known to those skilled in the art. The proximal impaction end of the instrument is then impacted with a mallet or similar instrument. Force from the mallet is transferred from the instrument to the shell as it is driven and permanently seated into the natural acetabulum. The shell should be driven into the acetabulum until the outer fixation spikes centrally engage into cancellous bone.

### Providing a Driving Instrument

The driving instrument generally includes a working section and a driving section. The working section has a handle adapted to be gripped with a hand. A proximal end of the working section can be adapted to connect to a manual T-handle or adapted to connect to a device for automated driving. The driving section is formed at an angle with respect the working section and includes a distal end. This distal end has a standard AO interface connection and is adapted to removeably connect to A driving bit. The driving instrument preferably is a fixed angled driver or a flexible angled driver.

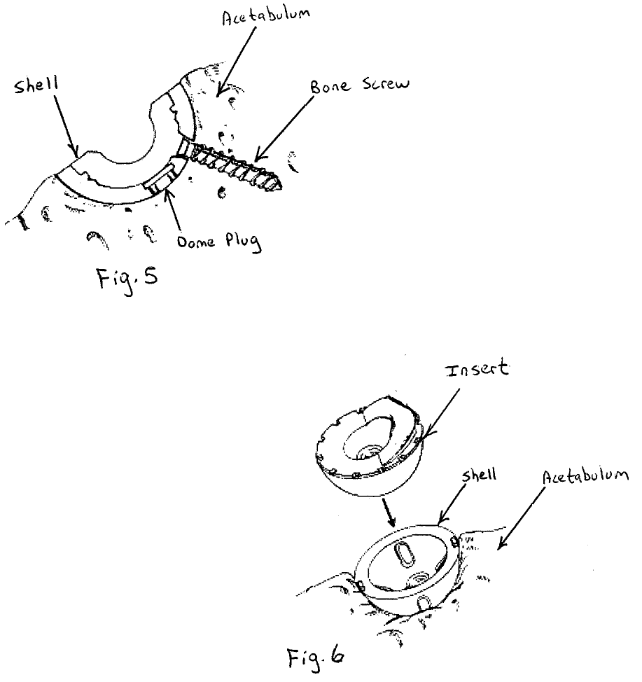

### Removing Screw-Hole Plugs (See FIG. 3)

The implant shell may be provided with screw-hole plugs, seals, or the like. In this instance, after the shell is properly seated in the acetabulum, one or more of the plugs may be removed with the driving bit. The driving instrument and attached driving bit are inserted through the incision, and the driving bit is engaged into the indentation of the plug. If the plugs are press-fit into the shell, then leverage is used to dislodge the screw-hole plug from the shell. If the plugs are screwed into the shell, then the driving bit is rotated to rotate the plug and remove it from the shell. The driving bit and attached screw-hole plug are removed from the surgical site.

### Installing Screw-Hole Plugs and a Dome Hole Plug (See FIG. 3)

The implant shell may be provided with screw-hole plugs or a dome plug that may be installed or inserted into the shell. Typically, these plugs have a head with a tool engaging recess. A threaded shaft extends from the head and is adapted to threadably engage a threaded bore in the acetabular shell.

The distal end of the driving bit is engaged with the plug. Specifically, the driving tip frictionally engages with the tool engaging recess in the plug. The plug and driving bit are then positioned into the surgical site so the threaded shaft on the plug engages the threaded bore in the acetabular shell. As the handle on the driving instrument is rotated, the driving bit simultaneously turns. The driving tip transfers torque to the plug and threads it into the threaded bore of the shell. Once the plug is fully threaded into the shell, the driving tip is disengaged from plug, and the driving bit is removed from the surgical site. At this time, another plug can be attached to the driving tip and the process is repeated as needed.

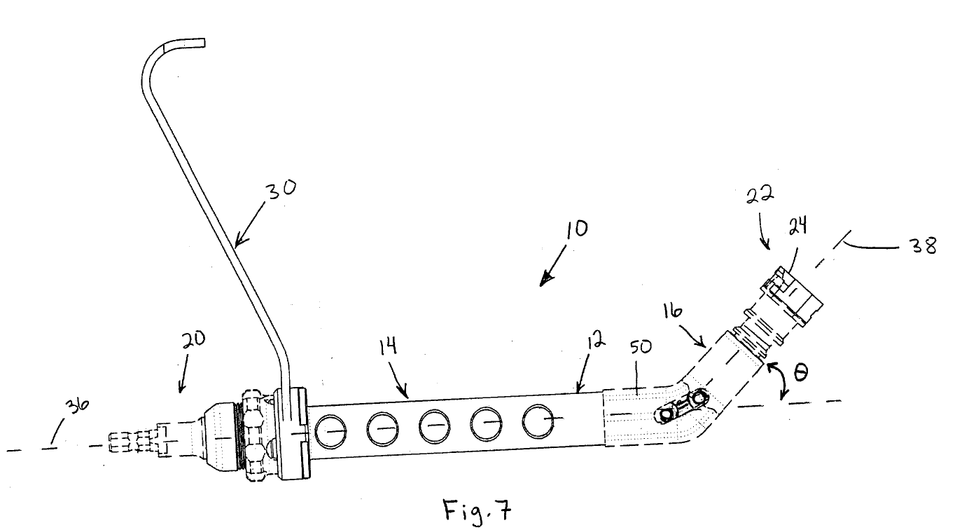

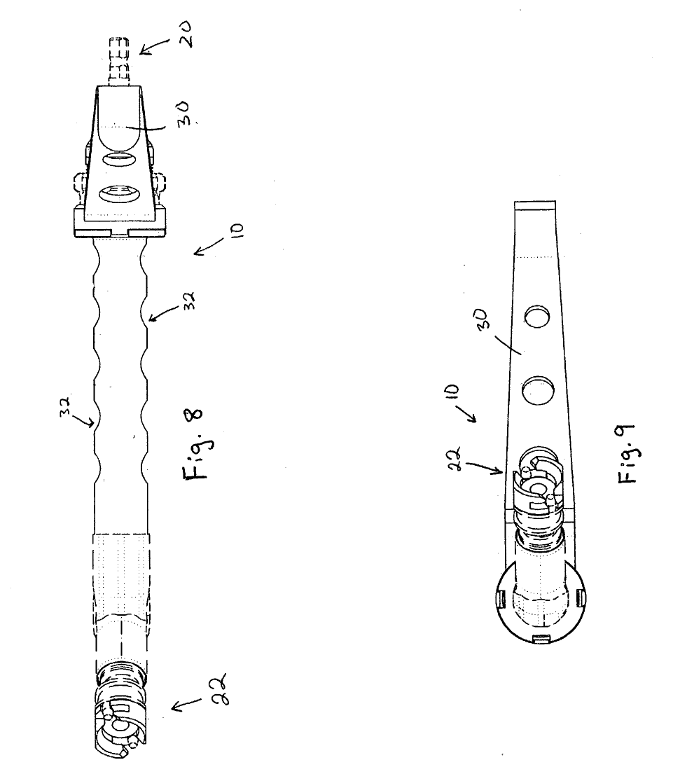

### Drilling Holes Through the Acetabular Shell for Bone Screws (See FIG. 4)

Next, a drill bit is provided, connected to a flexible driver, and positioned into the selected screw hole at an angle up to about 16°. As the hole or bore is drilled, care should be taken to protect the sciatic nerve and superior gluteal artery. A depth gauge may be inserted into the drilled holes to determine the depth for a corresponding bone screw. If desired, a tapping bit may be connected to the driver to tap the hole.

### Installing Bone Screws Through the Acetabular Shell (See FIG. 5)

A bone screw is inserted through the acetabular shell in a manner similar to inserting a screw-hole plug or dome plug. The driving tip of the driving bit is engaged with the bone screw. Specifically, the driving tip frictionally engages with a tool engaging recess in the bone screw. This recess may be provided as a Phillip's type recess, hexagonal recess, or other recesses known in the art. The bone screw and driving bit are then positioned into the surgical site so the threaded shaft on the bone screw passes through the screw-hole opening in the acetabular shell and into a drilled hole. As the handle on the driving instrument is rotated, the driving tip simultaneously turns. The driving tip transfers torque to the bone screw and drives it through the screw-hole opening and into adjacent cortical bone of the natural acetabulum. The bone screw should be seated into the countersunk holes of the shell so the acetabular insert can properly snap into the shell. Once the bone screw is fully threaded into the shell, the driving tip is disengaged from bone screw, and the driving bit is removed from the surgical site. At this time, another bone screw can be attached to the driving tip and the process repeated as needed.

### Inserting & Impacting Insert into Shell (See FIG. 6)

Various inserts known to those skilled in the art (such as standard, hooded, and protrusion inserts) can be inserted into the implant shell. Once the appropriate size and style insert is selected, the insert is connected to an instrument. The insert is positioned into the cavity of the shell and should be rotated to align with the antirotational pegs on the shell. A surgical mallet is used to strike the proximal end of the instrument to seat the insert into the shell.

### Closing Surgical Site

Once the insert is firmly connected to the shell, all instruments and devices are removed from the site. The acetabular shell and insert should now be properly positioned. Closure of the site may occur with well known techniques, such as posterior and anterior lateral approaches. Further, this disclosure will not discuss post-operative protocol or rehabilitation as such procedures are known in the art and tailored to meet the specific needs of the patient.

### Detailed Description of Acetabular Reamer

One important advantage of the present invention is that an angled acetabular reamer is used. This reamer is specifically designed and adapted to be used in minimally invasive surgical techniques for reaming the natural acetabulum of a patient.

Looking to FIGS. 7-9, the angled acetabular reamer 10 has a body 12 that includes two primary regions, a handle section 14 and a reaming section 16. Handle section 14 includes a proximal end 20 adapted to engage an automated driving or power mechanism (not shown). A distal end 22 of reaming section 16 includes a reaming end 24 adapted to engage an acetabular reamer. Proximal end 20 and distal end 22 can have configurations and connection mechanisms known in the art. Examples of such connections are more fully taught in U.S. Pat. No. 6,409,732 entitled “Tool Driver” and incorporated herein by reference.

Handle section 14 may also include a driving guide 30. Guide 30 has an elongated thin body adapted to be gripped with one hand. The guide can be permanently or removeably connected to the handle. Further, an exterior surface of the handle includes a plurality of rolling indentations 32 (see FIG. 8) adapted to be gripped with another hand.

A critical element of the present invention is that the reaming section 16 is disposed at an angle with respect to the handle section 14. As shown in FIG. 7, a longitudinal or central axis 36 extends through the handle section 14, and another longitudinal or central axis 38 extends through the reaming section 16. These axes form an angle θ. Preferably, this angle is about 45°. An elbow or sleeve 50 connects the handle and reaming sections.

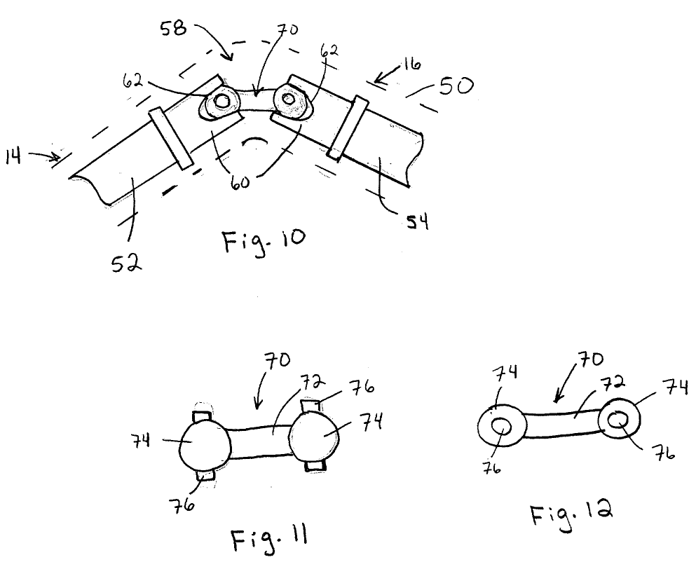

Looking to FIGS. 10-12, the inside of sleeve 50 is shown in more detail. A driving shaft 52 extends down through the interior of handle section 14, and a driven shaft 54 extends down through the interior of reaming section 16. These two shafts meet at the bow of the sleeve 50. Here, a coupling mechanism 58 connects the drive shaft 52 to the driven shaft 54. The driving and driven shafts have identical, blind socket cups 60 that have a spherical radius at the blind end. Each cup has at least two slots 62 cut into the sides, 180° apart. A coupler 70 shaped as a dogbone is positioned between the two shafts. This coupler 70 has a cylindrical center section 72 with a larger, spherical head or element 74 at each end. Two cross pins 76 protrude from the spherical elements. These cross pins are parallel and coplanar to each other, and mutually perpendicular but coplanar to a centerline axis of the center cylindrical section. The spherical elements at each end of the coupler rest in the blind socket cups in the end of each shaft. Each of the cross pins of the coupler engage the pair of slots cut into opposing sides of the shafts. During use, torque is transmitted from the slot sides of the driving shaft, to the cross pins in the coupler. Then, torque is passed through the coupler to the cross pins at the opposite end. The cross pins at the driven end of the coupler transmit the torque to the slots in the sides of the driven shaft. If the pairs of cross pins at each end of the coupler are coplanar to each other, the transferred rotary motion maintains a constant angular velocity throughout the mechanism. The coupler is retained along its axis by the blind socket cups in the ends of the shafts. The shafts are, in turn, retained axially in the sleeve by their respective self-retaining bearings.

One advantage of the present invention is that the coupler 70 minimizes the length of the driven shaft 54. As such, the reaming section can be relatively short. A long reaming section is not desired for reaming the acetabulum through a small MIS incision. Further, the shafts connect to the coupler so the instrument can operate with extremely low running torque.

The combination of the angle of the reaming section and orientation of the handle section provide the angled acetabular reamer with a configuration that is easy to use. Standard acetabular shell alignment tools are set up so that an alignment rod rises vertically from the wound while the patient is positioned to lie on the operating table. This orientation sets the abduction angle of the shell implant. The instrument of the present invention also performs this function. When the handle section is held vertically, it is in approximately the same orientation as the vertical alignment rod of a standard shell aligner. The standard shell aligner also has a rod or guide that sets the anteversion angle of the shell implant. The combination of these two angles result in a correct orientation of a shell implant relative to the patient's pelvis that will give the patient an acceptable range of motion. Anteversion control while reaming with the instrument can be at the desired anteversion angle while holding this angle with reference to the patient's coronal plane. This action will mimic the action of the shell aligner with the built-in anteversion reference rod. The surgeon can control anteversion while holding the main body of the acetabular reamer in a vertical position.

With the present invention, the surgeon can readily control, steer, and guide the depth of the reamer. Specifically, the driving guide 30 is adapted to help position the distal end 22 during use.

The instrument can be run in reverse so that bone chips, bone graft, or bone slurry can be applied to the acetabulum with a reamer cutter. The ramped teeth of the cutter act to pack the bone or bone graft materials into the porous cancellous bone of the patient's prepared acetabulum.

It should be emphasized that although the method of the present invention was described with a specific number and sequence of steps, these steps can be altered or omitted while other steps may be added without departing from the scope of the invention. As such, the specific steps discussed in the preferred embodiment of the present invention illustrate just one example of how to utilize the novel method and steps of the present invention. Further, although illustrative embodiments and methods have been shown and described, a wide range of modifications, changes, and substitutions is contemplated in the foregoing disclosure and in some instances, some features of the embodiments or steps of the method may be employed without a corresponding use of other features or steps. Accordingly, it is appropriate that the appended claims be construed broadly and in a manner consistent with the scope of the embodiments disclosed herein.

What is claimed is:

1. A method for using minimally invasive surgery to ream a natural acetabulum in a patient, comprising the steps of:
incising a hip with a minimally invasive incision;
providing an acetabular reaming instrument having a handle section and a reaming section, wherein the reaming section is disposed at an angle with respect to the handle section;
positioning the reaming section into the minimally invasive incision;
reaming the natural acetabulum with the reaming section;
removing the reaming section from the incision; and
closing the incision.

2. The method of claim 1 wherein the step of incising a hip creates the minimally invasive incision with a length of about 2½ inches to about 4 to 5 inches.

3. The method of claim 2 further comprising the step of forming the reaming section at an angle of about 45° with respect to the handle section.

4. The method of claim 1 further comprising the steps of:
providing the handle section with a drive shaft and the reaming section with a driven shaft; and
coupling the drive shaft to the driven shaft at an angle of about 45°.

5. The method of claim 4 further comprising the step of providing a coupler having a dogbone shape to couple the drive shaft to the driven shaft.

6. The method of claim 5 further comprising the step of housing the drive shaft, driven shaft, and coupler in an angled sleeve.

7. The method of claim 5 further comprising the step providing the coupler with two spherical heads connected with a straight cylindrical shaft.

8. The method of claim 7 further comprising the step of providing each spherical head with a cross pin.

9. A method for using minimally invasive surgery to position an acetabular shell into a natural acetabulum in a patient, comprising the steps of:
incising a hip with a minimally invasive incision;
providing an acetabular reaming instrument having a handle section with a drive shaft and a reaming section with a driven shaft;
coupling the drive shaft to the driven shaft so the driven shaft is at an angle with respect to the drive shaft;
providing an acetabular shell;
positioning the reaming section into the minimally invasive incision;
reaming the natural acetabulum with the reaming instrument;
removing the reaming instrument from the incision;
positioning the acetabular shell into the reamed acetabulum; and
closing the incision.

10. The method of claim 9 further comprising the step of providing the driven shaft at a 45 degree angle with respect to the drive shaft.

11. The method of claim 10 further comprising the step of coupling the driven shaft to the drive shaft with a coupler having a dogbone shape.

12. The method of claim 11 further comprising the step of providing the coupler with two spherical heads that are connected with a cylindrical body.

13. The method of claim 12 further comprising the step of connecting one end of the coupler to one end of the drive shaft and a second end of the coupler to one end of the driven shaft.

14. The method of claim 13 further comprising the step of providing the drive and driven shafts with spherical recesses to receive the coupler.

15. A method for using minimally invasive surgery to ream a natural acetabulum in a patient, comprising the steps of:
incising a hip with an incision having a length about 2½ inches to about 4 inches;
providing an angled acetabular reaming instrument having a handle section adapted to be gripped with a hand and a reaming section adapted to ream the acetabulum, wherein the reaming section is disposed at an angle with respect to the handle section;
positioning the reaming section into the incision;
reaming the natural acetabulum with the reaming section;
removing the reaming section from the incision; and
closing the incision.

16. The method of claim 15 further comprising the step of coupling the handle section at a 45 degree angle to the reaming section.

17. The method of claim 16 further comprising the step of providing a coupler to perform the step of coupling the handle section to the reaming section.

18. The method of claim 17 further comprising the step of providing the coupler with two spherical heads.

19. The method of claim 18 further comprising the step of providing a straight cylindrical body to couple the two spherical heads.

20. The method of claim 19 further comprising the step of providing each spherical head with a cross pin.
